(19) United States
(12) Patent Application Publication
(10) Pub. No.: US 2025/0206679 A1
(43) Pub. Date: Jun. 26, 2025

(54) NITROGEN-ENHANCED YEAST-BASED FERTILIZER

(71) Applicant: CHEMICAL EVOLUTION LTD., Calgary (CA)

(72) Inventors: Corynne O'FARRELL, Calgary (CA); Shravan TANUGULA, Edmonton (CA); Alejandra ENRIQUEZ, Calgary (CA); Markus WEISSENBERGER, Calgary (CA); Kyle G. WYNNYK, Calgary (CA)

(21) Appl. No.: 18/850,167

(22) PCT Filed: Mar. 23, 2023
(86) PCT No.: PCT/CA2023/050388

§ 371 Date: Sep. 24, 2024

(30) Foreign Application Priority Data
Mar. 25, 2022 (CA) 3153377

(51) Int. Cl. C05F 11/08; C05F 17/20; C12N 1/18

(52) U.S. Cl. CPC C05F 11/08; C05F 17/20; C12N 1/18

(57) ABSTRACT

A process to make a nitrogen-enhanced yeast-based fertilizer, said process comprising the steps of: —providing a live yeast (BSY) in solution; —exposing said live yeast to a nitrogen-containing compound to a carbohydrate and an enriched nitrogen source such as an amino acid, a protein or the like thereby creating an incubation mixture; —injecting air in to the incubation mixture so as to inhibit the production of ethanol; wherein said incubation mixture undergoes incubation for a period of time sufficient for said yeast to metabolize said nitrogen source and for said yeast to propagate and store the supplied nitrogen source in their vacuoles resulting in a nitrogen-fed yeast mixture; —hydrolyzing (or autolyzing) the resulting nitrogen-fed yeast mixture under specific conditions; and —optionally followed by a dehydration or evaporation step, to meet pre-determined specifications.

### FIELD OF THE INVENTION

The present invention is directed to a yeast-based fertilizing product, more specifically, a yeast-based fertilizing product having an enhanced nitrogen content.

### BACKGROUND OF THE INVENTION

Each year, agro-industrial activities produce large quantities of agro-industrial organic by-products resulting from activities as diverse as the wine industry, beer, meat production, flour, rice, dairy, etc. These activities generate large quantities of by-products, which is problematic due to their accumulation in the environment. Many of these by-products end up in municipal landfills or wastewater treatment plants, where they create serious environmental and equipment problems due to microbial decomposition, contamination, and leachate production. From an economic standpoint, there is an additional cost related to the handling of solid waste and its incineration, leading to large amounts of greenhouse gas emissions. There is constant and growing pressure from political groups, as well as environmental entities, to take steps to reduce pollution.

In spite of their diverse origin, agro-industrial by-products are characterized by high organic matter content, including high amounts of protein. One of the main areas of use of waste protein material is in the production of nutritional products for plants. Waste protein materials are used as a nitrogen source for soil in the form of fertilizers. Fertilizers are organic or inorganic substances, of natural or synthetic origin, that are used to enrich soil and provide plants with one or more nutritional elements essential for plant development.

Insufficient supply of nitrogen to crops results in poor overall growth smaller leaves, a reduction in chlorophyll production and chloroplast development, which leads to chlorosis of the entire crop. Plant growth can be stunted because of the lack of nitrogen.

Organic fertilizers are not only a great source of nutrients for plants, but they also provide organic matter to the soil that may, in some instances of poor soil quality, contribute to the physical, chemical, and biological properties of the soil. In the food and beverage industry, yeast is used for baking, alcohol production, feed protein, health food raw materials, single-cell protein, vitamins, and nucleic acid-related substances. Recently, there has been a renewed interest in using yeast as a fertilizing agent in the agro-industrial sector.

One of the abundant sources for organic matter is Brewer's Spent Yeast (BSY) (also known as residual yeast or surplus yeast). This refers to a prevalent by-product of the brewing industry, created when the yeast used in fermentation is no longer useful and must be disposed of BSY is mainly composed of the yeast strain Saccharomyces cerevisiae, although hundreds of strains are known to be involved in the alcohol fermentation process. Yeast cells contain a wide range of different functional components including peptides, amino acids, polyphenols, carotenoids and flavonoids, which impart bioactive properties to the yeast extract once extracted from the cell. Yeast degradation yields a wide variety of compounds, vitamins, and minerals. It may yield upwards of 40% protein, less than 1% in fat, close to 40% of carbohydrates with various other components rounding out the remaining portion. Among the protein portion, amino acids have been analyzed to account for varying amounts (based on the yeast strain) ranging from close to 0.4% to upwards of 7%. These amino acids include: Lysine (Lys); Methionine (Met); Tryptophan (Trp); Arginine (Arg); Histidine (His); Isoleucine (Ile); Leucine (Leu); Phenylalanine (Phe); Threonine (Thr); Valine (Val); Glycine (Gly); Cystine (Cys); Tyrosine (Tyr); Alanine (Ala); Serine (Ser); Aspartic Acid (Asp); and Glutamic Acid (Glu).

Preferably, the discarded BSY consists of yeasts and by-products from the alcoholic fermentation of barley malt. It consists of minerals, traces of fatty acids, and carbohydrates. Some of the minerals present include: potassium, sulfur, magnesium, calcium or sodium. Traces of unsaturated fatty acids include: lecithins, and cephalins. Carbohydrates such as: glycogen, trehalose, glucans or mannans, ethyl alcohol, carbon dioxide, traces of esters, aldehydes, ketones and higher alcohols, etc. These residues may be suspended in beer as they leave the industrial plant, without the need for drying or concentration, or be in solid form once the remaining beer has been removed. The remaining beer can be separated by any conventional method such as filtration, sedimentation or centrifugation.

Many patent applications and patents discuss various uses of yeasts for such applications. A few of them are set out below to provide an overview of the field. Hungarian patent document HU 9902060 discloses the composition of an aqueous fertilizer for the leaves and roots of plants containing yeast of the genus Saccharomyces, trace elements, complexing agents, buffering agents and other nutrients such as amino acids, humic acids, enzymes, carbohydrate sources, etc.

Chinese patent application CN19191800 describes a nutrient for animals and plants that is prepared by diluting Saccharomyces cerevisiae sludge in water until an emulsion is obtained; mixing this with papain, neutral proteases and sodium chloride; hydrolyzing the mixture afterwards; inactivating enzymes; and finally, concentrating or drying the product obtained. The final product contains a large amount of nutrients.

Chinese patent application CN 191 1870 describes a plant nutrient that improves soil microorganisms, promotes plant growth, increases fertilizer utilization rates, and increases plant resistance to disease and stress. This nutrient is composed of Saccharomyces cerevisiae, Lactobacillus plantarum, Lactobacillus acidophilus, and other components such as potato or coffee derivatives, glucose, peptone, magnesium and manganese sulfates, dipotassium hydrogen phosphate, and sodium chloride.

US patent application 2003/022357 discloses a biological fertilizer containing magnetically activated yeast cells of the genus Saccharomyces and sludge from wastewater treatment or storage.

US patent application no. 2002/187900 discloses a biological fertilizer comprised of electromagnetically activated yeast cells of the genus Saccharomyces and cattle manure.

US patent application no. 2009/0173122A1 discloses a soluble, liquid or dry fertilizer for application to a plant or soil that is grown or farmed as “organic” as defined under the USDA National Organic Program Rule. The fertilizer is produced from distiller's yeast from beer and/or alcohol production. The yeast cells are autolyzed using heat and the autolysates are separated by centrifugation into insoluble cell walls and cellular plasma. The plasma is concentrated by evaporation into the fertilizer. It also stated that the fertilizer may be further processed by proteolytic enzyme (protease) lysis to produce smaller-sized, soluble, nitrogen-containing compounds including protein, peptides, amino acids, amines and ammonia. The fertilizer has a solids content between ten and sixty-five percent, a total protein content of at least ten percent and up to eighty-five percent, a total Nitrogen content between one and fourteen percent, and a pH between 2.5 and 10.

In light of the state of the art, there exists a need to improve the production of organic fertilizers using yeast-based starting materials. This is true especially when considering the fact that yeast waste is underused and could benefit the agriculture sector in a much more substantial manner.

### SUMMARY OF THE INVENTION

According to an aspect of the present invention, there is provided a process for obtaining organic extracts from residues of the beer industry that can be used as bio-stimulants and biofertilizers in agriculture, preferably organic farming.

According to a first aspect of the present invention, there is provided a process to make a nitrogen-enhanced yeast-based fertilizer, said process comprising the steps of:
providing a live yeast (BSY) in solution;
exposing said live yeast to a nitrogen-containing compound or an enriched nitrogen source such as an amino acid, a protein or the like and a carbohydrate source thereby creating an incubation mixture;
injecting air into the incubation mixture so as to minimize and/or substantially inhibit the production of ethanol;
   wherein said incubation mixture undergoes incubation for a period of time sufficient for said yeast to metabolize said nitrogen source and for said yeast to propagate and store the supplied nitrogen source in their vacuoles resulting in a nitrogen-fed yeast mixture;
hydrolyzing (or autolyzing) the resulting nitrogen-fed yeast mixture under specific conditions; and
optionally followed by a dehydration or evaporation step, to meet pre-determined specifications.

Preferably, the nitrogen-containing compound is any naturally occurring organic protein source like an amino acid or peptide and/or a mixture of amino acids or peptides and/or an amine salt thereof.

After undergoing lysis, yeast extracts contain practically all the hydrolyzed protein in the form of free amino acids, oligopeptides and other peptides of greater molecular weight and, in addition, practically all the nutrients of the starting residue. These extracts are highly prized and have a great potential as biofertilizers and bio-stimulants. Moreover, they are highly sought after as they fulfill the requirements to be used in organic farming settings. Allowing brewer's spent yeast (BSY) to propagate under nitrogen rich conditions and accumulate the wort nitrogen content for propagation and storage in its vacuoles.

According to a preferred embodiment of the present invention, the nitrogen-containing compound is an inorganic amine salt.

Preferably, the amino acid is selected from the group consisting of Lysine (Lys); Methionine (Met); Tryptophan (Trp); Arginine (Arg); Histidine (His); Isoleucine (Ile); Leucine (Leu); Phenylalanine (Phe); Threonine (Thr); Valine (Val); Glycine (Gly); Cystine (Cys); Tyrosine (Tyr); Alanine (Ala); Glutamine (Gln); Asparagine (Asn); Proline (Pro); Serine (Ser); Aspartic Acid (Asp); and Glutamic Acid (Glu); and a combination thereof and/or salts thereof.

According to a preferred embodiment of the present invention, the incubation period has a duration ranging from 12 to 48 hours. Preferably, the incubation period has a duration ranging from 20 to 36 hours.

According to a preferred embodiment of the present invention, the feed rate and the feed quantities vary for each individual fertilizer product.

According to a preferred embodiment of the present invention, the nitrogen source added to the live yeast in solution is equivalent to between 0.5 and 20% of the total composition. Preferably, the nitrogen source added to the live yeast in solution is equivalent to between 0.5 and 15% of the total composition. More preferably, the nitrogen source added to the live yeast in solution is equivalent to between 0.5 and 10% of the total composition.

According to a preferred embodiment of the present invention, the yeast (BSY) dry content ranges between 0-30 wt. % of the total composition prior to optional evaporation. Preferably, the yeast dry content ranges between 7-13 wt. % of the total composition.

According to another aspect of the present invention, there is provided an enhanced yeast extract made by a process comprising a step of exposing yeast to added nutrients prior to a lysis of said yeast.

Preferably, the process further comprises a step of exposing yeast to added nutrients prior to lysis of said yeast.

According to a first aspect of the present invention, there is provided a fertilizer comprising a yeast extract enhanced with the addition of at least one nitrogen-containing compound prior to a yeast incubation step. According to a preferred embodiment of the present invention, the amino acid is selected from the group consisting of alanine; arginine; asparagine; aspartic acid; cysteine; glutamic acid; glutamine; glycine; histidine; isoleucine; leucine; lysine; methionine; phenylalanine; proline; serine; threonine; tryptophan; tyrosine; and valine or a combination thereof. According to another preferred embodiment of the present invention, the nitrogen-containing compound is an inorganic amine salt. Preferably, the nitrogen content in the fertilizer ranges between 1 and 7 wt. %.

### BRIEF DESCRIPTION OF THE FIGURES

The invention may be more completely understood in consideration of the following description of various embodiments of the invention in connection with the accompanying figures, in which:

FIG. 1 is a graphical representation of the shoot weight of pea plants (control group, treated fish fertilizer group and treated with a composition obtained according to a preferred embodiment of a process of the present invention;

FIG. 2 is a graphical representation of the dry root weight of pea plants (control group, treated fish fertilizer group and treated with a composition obtained according to a preferred embodiment of a process of the present invention;

FIG. 3 is a graphical representation of the root to shoot ratio of pea plants (control group, treated fish fertilizer group and treated with a composition obtained according to a preferred embodiment of a process of the present invention;

FIG. 4 is a graphical representation of the nitrogen content in leaves of pea plants (control group, treated fish fertilizer group and treated with a composition obtained according to a preferred embodiment of a process of the present invention;

FIG. 5 is a graphical representation of the shoot weight of basil plants (control group, treated fish fertilizer group and treated with a composition obtained according to a preferred embodiment of a process of the present invention;

FIG. 6 is a graphical representation of the shoot height of basil plants (control group, treated fish fertilizer group and treated with a composition obtained according to a preferred embodiment of a process of the present invention;

FIG. 7 is a graphical representation of the dry root weight of basil plants (control group, treated fish fertilizer group and treated with a composition obtained according to a preferred embodiment of a process of the present invention;

FIG. 8 is a graphical representation of the root to shoot ratio of basil plants (control group, treated fish fertilizer group and treated with a composition obtained according to a preferred embodiment of a process of the present invention;

FIG. 9 is a graphical representation of the nitrogen content in leaves of basil plants (control group, treated fish fertilizer group and treated with a composition obtained according to a preferred embodiment of a process of the present invention;

FIG. 10 is a graphical representation of the shoot weight of eggplants (control group, treated fish fertilizer group and treated with a composition obtained according to a preferred embodiment of a process of the present invention;

FIG. 11 is a graphical representation of the dry root weight of eggplants (control group, treated fish fertilizer group and treated with a composition obtained according to a preferred embodiment of a process of the present invention;

FIG. 12 is a graphical representation of the root to shoot ratio of eggplants (control group, treated fish fertilizer group and treated with a composition obtained according to a preferred embodiment of a process of the present invention;

FIG. 13 is a graphical representation of the nitrogen content in leaves of eggplants (control group, treated fish fertilizer group and treated with a composition obtained according to a preferred embodiment of a process of the present invention.

### DETAILED DESCRIPTION OF AN EMBODIMENT OF THE PRESENT INVENTION

The description that follows, and the embodiments described therein, is provided by way of illustration of an example, or examples, of particular embodiments of the principles of the present invention. These examples are provided for the purposes of explanation, and not limitation, of those principles and of the invention.

Nitrogen is a basic constituent of proteins, nucleic acids, cell components, etc. and, therefore, allows the development of the metabolic activity of plants and microorganisms. Amino acids, oligopeptides and peptides of low molecular weight constitute nutritious substances which can be taken up and assimilated easily by plants. Fertilizers containing these compounds can be applied on the leaves of plants or to the root system where they will be transported to the flowers, fruits, etc. to provide essential nutrients for the development of the plant. According to a preferred embodiment of the present invention, there is provided a process to make a nitrogen-enhanced yeast-based fertilizer, which is to be understood as being a fertilizer which contains a high quantity of nitrogen compared to other fertilizers obtained through the use of yeast.

BSY is a by-product of the brewing industry. According to a preferred embodiment of the present invention, a quantity of nitrogen supplement that can be a protein or a mixture of amino acids is fed to the BSY. The yeast is left for a period of approximately 12-48 hours to incubate under aerobic conditions. During that period, the yeast extracts the nitrogen in the amino acids to create further proteins which are readily bioavailable for plants and the like. At the end of the 12-48 hours, the yeast is hydrolyzed. After lysis, if required, the hydrolyzed yeast is administered an additional mixture of amino acids to further increase the nitrogen content in the liquid solution of hydrolyzed yeast.

The liquid fertilizer is now ready to be used. The pH of the solution is between 4.0-7.0. The nitrogen content is about 1-14%. These amounts can vary depending on the quantity of nitrogen sources added during the first and second addition. Moreover, the type of nitrogen source used during the addition will also have a direct impact on the ultimate nitrogen content of the liquid fertilizer.

According to a preferred embodiment of the present invention, there is provided a process to make a nitrogen-enhanced yeast-based fertilizer. According to a preferred embodiment of the present invention, there is provided a nitrogen-enhanced yeast-based fertilizer which is organic or can be labelled as organic. Preferably, the nitrogen-enhanced yeast-based fertilizer is in liquid form and can be dispensed on or near plants, or foliage, or roots. Preferably, applying such a composition will allow nitrogen to be readily available for uptake by plants.

According to a preferred embodiment of the present invention, the compounds that may be used to increase the nitrogen content of the yeast include: amino acids, peptides, heterocycles, substituted amines, other industrial by-products, and yeast extracts. Preferably, the compounds that are amino acids to be selected from the group consisting of: alanine; arginine; asparagine; aspartic acid; cysteine; glutamic acid; glutamine; glycine; histidine; isoleucine; leucine; lysine; methionine; phenylalanine; proline; serine; threonine; tryptophan; tyrosine; and valine.

According to a preferred embodiment of the present invention, the amino acids can be added as is or as a salt thereof. Preferably, the amino acids used have high N:C ratio, such as: lysine, asparagine, tryptophan, and glutamine (each have 2 nitrogen atoms); arginine (4 nitrogen atoms); histidine (3 nitrogen atoms) are particularly preferred. Most preferred, is lysine sulfate. Lysine sulfate is commonly produced by bacterial fermentation and is typically used as a feed additive to farm animals, poultry, and fish. As such, it is considerably cheaper than other sources of lysine and moreover, it readily dissolves in water.

According to a preferred embodiment of the present invention, the liquid fertilizer solution may be spray dried into a powder, oven dried or granulated. According to a preferred embodiment of the present invention, the liquid fertilizer can also be concentrated by evaporation or diluted to a more soluble state.

It is noteworthy to mention that according to a preferred embodiment of the present invention the conversion of carbohydrate source into ethanol is avoided as much as possible, as the target products are high nitrogen-containing compounds. In aerobic respiration, the yeast converts carbon sources to CO₂. Removal of carbon and oxygen through this results in an increase in the concentration of nitrogen in the system.

The insoluble, solid yeast cell walls are removed, and the water/yeast mix is then concentrated. Removal of the insoluble solids may be done using filters, decanters or centrifuges. Concentration may be achieved by using equipment such as evaporators, or membrane filters.

According to a preferred embodiment of the present invention, the resulting yeast fertilizer product has the following characteristics: solids content between 10 and 80% solids on a weight-to-weight basis; a total Nitrogen content between 1 and 12% on a weight-to-weight basis; and a final pH of between 4.0 and 7.0.

According to a preferred embodiment of the present invention, the resulting fertilizer may be dried into a soluble solid. Preferably, the fertilizer is stable at normal environmental temperatures and requires no special handling.

The following examples illustrate the invention and should not be considered as limiting its scope:
obtaining brewers spent yeast (BSY) followed by
an incubation step for 12-48 hours in the presence of added nutrients under aerobic conditions followed by
a step of lysis of the yeast under specific conditions; and
optionally followed by a dehydration or evaporation step, if required, to meet predetermined specifications.

According to a preferred embodiment of the present invention, there is provided a process for obtaining an organic extract from the spent yeast used in the brewing industry or any industry that produces alcohol by fermentation or yeast grown under aerobic conditions in a specific controlled growth media comprising the following steps:
1) brewer's spent yeast (BSY) can be collected in its stationary phase, where it flocculates after reaching a certain concentration in the wort, which could be obtained as a slurry with 5-30% solid content; preferably between 12-17%;
2) supplementing BSY with 2-15% wt. of a specially designed wide range of organic or inorganic nitrogen rich substances to promote exponential growth;
3) supplementing BSY with up to 10% wt. of a wide range of carbohydrate sources to allow growth under aerobic conditions and accumulation of excess wort nitrogen;
4) lysis of BSY under moderate conditions after feeding the yeast with nitrogen rich feed; and
5) optionally, depending on the application of the invention, the BSY product undergoes further processing.

According to a particular embodiment of the process of the invention, the organic yeast fertilizer manufacturing method uses BSY with a solid content of 5-30%. Preferably, a source of nitrogen and carbohydrate source is added to the live BSY to bring the yeast from the stationary phase to the exponential phase, where the yeast is incubated under aerobic conditions for a period of 12-48 hours. After incubation, autolysis is induced. The resulting product is separated and concentrated into desired fertilizer product.

In the context of the present invention the expression “in a single container” (one-pot) refers to the fact that the process is carried out without intermediate stages of separation so that the nitrogen and mass yield is as close to 80% as possible by eliminating any potential loss during handling.

As will be understood by the person skilled in the art, the expression “biofertilizers and bio-stimulants” refers to compounds with the capacity to stimulate the growth and development of plants and crops, as well as to increase and enhance the microbiological activity of the soil.

According to a preferred embodiment, the yeast vacuolar proteases Cerevisin (EC 3.4.21.48, yeast proteinase B, proteinase yscB, baker's yeast proteinase B, brewer's yeast proteinase, peptidase beta), which are active at two different pH ranges, are used to break down the proteins obtained during autolysis of yeast cells from higher molecular weight proteins to lower molecular weight peptides. Thus, avoiding an external addition of proteases like papain to denature the complex proteins. Cerevisin, which is a yeast internal protease, is used to break down the externally added protein like gelatin, pepsin etc and make the nitrogen more available for plants.

According to a preferred embodiment of the present invention, if the yeast extract contains turbidity due to incomplete yeast protease activity the product can be filtered, settled out or an external enzyme can be added for the lysis of peptides to more soluble amino acids. In the most preferred embodiment, the enzyme used is papain.

The conditions of pressure, temperature, pH, and time of lysis will be those in which the maximum activity of the enzyme is achieved. Thus, in another particular embodiment of the process of the invention, the lysis of step is carried out at a temperature of 35-55° C. and a pH of 3-11 for a time of 2-48 h. According to a more preferred embodiment of the present invention, the lysis is carried out at a temperature of 45-55° C. and a pH of 4.0-7.0 for a time of 24-48 h.

According to a particular embodiment of the process of the invention, during the lysis the pH value is kept constant by the addition of pH buffers of various chemical backgrounds.

In another aspect of the invention, the use of organic extracts previously described in agriculture and animal feed is provided. More particularly, the organic extracts of the invention can be used as a bio-stimulant and biofertilizer in organic farming given its special composition of free amino acids, oligopeptides and low molecular weight peptides. It can also be used as a nutritional additive of high added value for animal feed for livestock (bovine, sheep, goats, etc.) aquaculture, or for domestic or companion animals.

According to a preferred embodiment of the present invention, the method of applying the yeast extract to an agricultural crop can be done by carrying out any conventional technique such as, for example, direct application in the soil, by foliar route, or by fertigation.

On the other hand, the organic extract of the invention may alternatively be subjected to subsequent stages of concentration and/or separation for stabilization.

According to a preferred embodiment of the present invention, the soluble yeast extract can be separated from an insoluble solid phase containing the insoluble organic matter remaining after the yeast lysis step. This separation can be carried out by any conventional method of the prior art, such as, for example, by filtration or centrifugation using a decanter or other suitable industrial device.

According to a preferred embodiment of the present invention, the yeast product can be used as it contains substantially all the hydrolyzed starting protein, at least more than 90% by weight, and therefore has a composition that also makes it suitable for use in agricultural and livestock applications.

According to a preferred embodiment of the present invention the yeast extract can be used as a bio-stimulant and biofertilizer in organic farming given its special composition of free amino acids, oligopeptides and low molecular weight peptides.

According to a preferred embodiment of the present invention, yeast extract can be subjected to concentration. Preferably, the concentrated soluble organic extract of the invention has at least 40% by weight dry matter, preferably at least 50% by weight dry matter and, more preferably, 50-65% by weight dry matter. This concentration can be carried out by any conventional method of the prior art, such as, for example, by heating and vacuum using a rotary evaporator with a thermostatic bath or a reverse osmosis system, for example, or another suitable device.

According to a preferred embodiment of the present invention, the concentrated organic yeast product and the concentrated soluble organic yeast extract are extracts with a composition that also makes them suitable for use in agricultural and livestock applications.

According to a preferred embodiment of the present invention, the yeast product can be used as a nutritional additive of high added value for animal feed, more particularly, in animal feed in livestock (bovine, sheep, goats, etc.) or aquaculture, or for domestic or companion animals.

### Experiments Based on Brewer's Spent Yeast

According to a preferred embodiment of the present invention, BSY is obtained from a local brewery which is a waste product for most of the breweries. Yeast (mainly S. Cerevisiae strain) has four growth phases that are lag phase, exponential phase, stationary phase and death phase.

Fresh BSY when obtained from the brewery is generally in a stationary phase and contains wort, which is mostly starch, and small amounts of fermentable carbohydrate sources (contents varies based on the fermentation conditions and supply chain delay). The nitrogen content for dry BSY was measured to be in the range of 7 to 9%.

According to a preferred embodiment of the present invention, the live BSY is incubated in the presence of nutrients and under aerobic conditions. The aerobic conditions can be achieved through a variety of ways including, but not limited to, the bubbling (or injection) of air into the vessel where the yeast, sugar (source of carbohydrate) and source of nitrogen are mixed together. Having an open top vessel for the incubation may partially achieve this goal but may not be sufficient to inhibit the production of ethanol. The obtained BSY, which is in stationary phase, which is generally a 5-30% slurry, preferably 12-17% (yeast+solids) and up to 95% liquid (water), is revitalized by the addition of some ingredients and the brew is brought back to the exponential phase, where the yeast multiplies under aerobic conditions to reduce ethanol production and increase cell growth in presence of a carbohydrate source and extra nitrogen source for next 12-48 hours. The nitrogen is consumed by the yeast partly for propagation and partly it is stored in the vacuoles of yeast cells.

This step increases the cell count and thus, contributes to the total nitrogen content. The vitamins and minerals required by the yeast are supplied from the spent liquid wort. This would eventually run the wort to dry conditions by using all available carbohydrate sources and nutrients in presences of excess nitrogen.

According to a preferred embodiment of the present invention, subsequent to the incubation of the yeast follows a step of lysis of the yeast. Preferably, after 24 hours of revitalized exponential growth phase, the yeast enters back to the stationary phase, after which the broth is heated to 45-50° C. for next 24-48 hours, where the yeast internal enzymes promote autolysis of the cells releasing proteases into the liquid. As BSY contains about 48% protein content, the proteases act upon the proteins and cleave them and generates smaller amino acids. The same proteases also dissociate any leftover proteins like gelatin, that is not completely consumed during the growth that are added to the broth. Thus, increasing the overall bioavailable nitrogen content. At this stage, the broth is separated into two phases, the top layer is the water-soluble phase with dissolved proteins, amino acids, nucleic acids etc. and the bottom is the flocculated insoluble layer or cell walls and carbohydrate wort from the BSY-wort mixture.

According to a preferred embodiment of the present invention, subsequent to the lysis step the top layer is collected. This fraction when collected has 0.2 to 4% nitrogen. Preferably, up to 60-66% of water is removed and the liquid is concentrated to obtain higher nitrogen content (up to 12%), phosphorus and potassium contents up to 1% respectively, as a natural or totally organic source of liquid fertilizer. According to a preferred embodiment of the present invention, the above-mentioned liquid fertilizer can be further formulated by the addition of excess nitrogen, phosphorus and potassium to obtain any required concentrations of NPK values.

### EXPERIMENTAL

Various experiments were carried out to determine the impact of using yeast on Nitrogen testing is carried out using the Dumas method AOAC 993.13 and the Kjeldahl method AOAC 962.10. The results of those experiments are compiled in Table 1.

### Experiment Set-1 (081621-A)

### a) BSY

The yeast+wort from BSY was thoroughly mixed and a portion of mixture was weighed and dried in an oven at 45° C. for 48 h. The dry yeast was weighed to record the loss on drying. The loss on drying was 83% of total weight. The dry yeast was sent for total Nitrogen analysis where it was determined to have a content of 6.7%.

### b) Incubation

It was not incubated in the presence of any supplements

### c) Lysis of Yeast

The yeast was hydrolyzed at 45° C. for 24 hours and the hydrolysate was tested for total nitrogen content and obtained 0.68% of total Nitrogen in the liquid portion. The insoluble cell wall layer was dried in an oven for 48 h at 45° C. and the dry portion was sent for total nitrogen analysis to obtain a nitrogen content of 2.2%.

Raw Yeast hydrolysate was concentrated by removing 66% of the water. In sample 081621-A/1, the nitrogen content was measured to be 1% (as per the test method there is a ±0.1% error). In sample 081621-A/2, upon the addition of 5% of lysine monohydrochloride, the resulting nitrogen concentration was measured to be 2%.

It was possible to achieve a maximum extraction amount of nitrogen at 15-17% loading into the extract, a portion of it was lost to the cell wall.

### Experiment Set-2 (081621-B/1 and 081621-B/2)

### a) BSY

The yeast and wort from BSY were mixed well and a portion of the mixture was weighed and dried in an oven at 45° C. for 48 h. The dry yeast was weighed to record the loss on drying. The loss on drying was 85% of the total weight. The dry yeast was sent for total Nitrogen analysis to obtain a nitrogen content of 7.1%.

### b) Incubation in Presence of Nutrients

BSY was incubated with 2.6% of amine rich media that included amino acids, ammonium sulfate and a glucose source (0.5% Gly., 0.8% Lys., 0.5% His., 0.8% (NH₄)₂SO₄ and 1% Glucose). The BSY was grown for 48 h before lysis (081621-B) and the hydrolysate was concentrated by evaporation of 60% water. In sample 081621-B/1, the total nitrogen analysis was done on the concentrate which showed a nitrogen content of 2.2%. In sample 081621-B/2, to the above concentrate 5% Lysine HCl was added and yielded a total nitrogen content of 3.4%.

### c) Lysis of Yeast

The yeast was hydrolyzed at 45-55° C. for 24 hours and the hydrolysate was tested for total nitrogen content. Results showed 0.82% total Nitrogen in the liquid portion. The yeast with added lysine was hydrolyzed to obtain a result of 1.22% Nitrogen.

### d) Liquid Fertilizer

The water from the yeast hydrolysate (control) was evaporated and the liquid was concentrated. About 66% of the water was evaporated and the concentrate was measured for nitrogen content to obtain 2% of total Nitrogen. Similarly, 66% of water was evaporated from the lysine hydrolysate and measured for total nitrogen content to obtain 4% of total Nitrogen.

### Experiment Set-3 (062321-A/4/5)

### a) BSY

The yeast and wort from BSY were mixed well and a portion of mixture was weighed and dried in an oven at 45° C. for 48 h. The dry yeast was weighed to record the loss on drying. The loss on drying was 75% of the total weight.

### Results Experiment #3:

Yeast was fed with 2.5% lysine monohydrochloride, which is an addition of 0.38% nitrogen, to obtain a result of 1.22% Nitrogen. When the yeast was concentrated to one third its original volume (66% of volume as water was removed) the nitrogen concentration increased to 4%.

It was noted that an addition of 2.5% lysine to the initial feed (BSY) followed by lysis increased the final nitrogen content of the hydrolyzed yeast extract.

The expected value of nitrogen due to the reduction in volume by 66% was 3.6% (±0.1%) N. The obtained value was 3.9% (±0.1%), almost a 0.3% increase in nitrogen content (2.5% Lys. HCl).

### b) Incubation in the Presence of Nutrients

An attempt was made to attain calculated concentration of nitrogen by adding it to the concentrated hydrolysate, it was observed that it is more productive to add the supplement to the live yeast to attain the desired concentrations.

c) Lysis of yeast: The yeast was hydrolyzed at 45° C. for 24-48 hours.
d) Liquid fertilizer: The water from the yeast hydrolysate (control) was evaporated and the liquid was concentrated. About 60-66% of water was evaporated and the concentrate was measured for nitrogen content to obtain 1.0-2.5% of total N on control.

Table 1 provides a summary of the results of an experiment carried out using live yeast and a variety of amino acids.

TABLE 1
Summary of experiments

| | | | Expected N % | |
|---|---|---|---|---|
| | Sample | | (calculated based | |
| | Number | Modification | on previous results) | Obtained N % |
| Experiment #1 | 081621-A/1 | Raw Yeast hydrolysate 48 h at 45° C. (081621-A), | around 2% | 1% |
| (control) | | concentrated to 33%-Neat | | |
| Experiment #1 | 081621-A/2 | Raw Yeast hydrolysate 48 h at 45° C. (081621-A), | 1% + 0.77% | 2% |
| | | concentrated to 33%-and added 5% Lys. HCl after | | |
| | | concentration. | | |
| Experiment #2 | 081621-B/1 | Yeast supplemented with amino acids (0.5% Gly., 0.8% | 0.8 + 0.93 | 2.2% |
| | | Lys., 0.5% His., 0.8% (NH₄)₂ SO₄ and 1% | | |
| | | Glucose) and grown for 48 h before lysis (081621-B) | | |
| | | and the hydrolysate concentrated to 30%. | | |
| Experiment #2 | 081621-B/2 | Yeast supplemented with amino acids and grown for | 2.2 + 0.75 | 3.44% |
| | | 48 h before lysis (081621-B) and the hydrolysate | | |
| | | concentrated to 30%. Added extra 5% Lys. HCl to the | | |
| | | concentrate. | | |
| Experiment #3 | 062321-A/4 | Raw Yeast hydrolysate 48 h at 45° C. (062321-A), | 0.66 + 1.125% | 1.81% |
| | | concentrated to 50%, added 7.5% Lys. HCl. To the | | |
| | | concentrate. | | |
| Experiment #3 | 062321-A/5 | Raw Yeast hydrolysate 48 h at 45° C. (062321-A), | 0.66% + 1.5% | 2.3% |
| | | concentrated to 50%, added 10% Lys. HCl. To the | | |
| | | concentrate. | | |
| Experiment #3 | 081621-D/2 | 081621 B (30% concentrate) and added 15% Lys. | 2.20 + 2.25% | 4.18% |
| | | HCl. | | |

According to a preferred embodiment of the present invention, the soluble liquid or dry fertilizer for application to a plant or soil can be certified as “organic” as defined under the USDA National Organic Program Rule. According to a preferred embodiment of the present invention, at the root of the process, yeast is used. Preferably, brewer's spent yeast, which is a by-product of the brewery industry and ethanol production by fermentation. Preferably, the yeast-based fertilizer is produced by addition of one or more nitrogenous compounds in order to increase the nitrogen content of the yeast prior to lysis. Subsequent lysis of the yeast will convert proteins present in the yeast to generate small-size, water-soluble, nitrogen-containing compounds including protein, peptides, amino acids, amines, and ammonia.

According to a preferred embodiment of the present invention, the yeast product has a solids content between 5 and 40 percent. Preferably, the solids content ranges from 15 to 30 percent.

According to a preferred embodiment of the present invention, the yeast-based fertilizer has a total nitrogen content between 1 and 12 percent.

According to a preferred embodiment of the present invention, the yeast-based fertilizer has a pH between 3.5 and 7.0.

According to a preferred embodiment of the present invention, the yeast-based fertilizer is generated by heating the yeast at around 45-55° C. for a period of 24-48 hours to induce autolysis of the yeast cells.

Additional experiments were carried to out to compare the impact of yeast incubation on nitrogen extract. Table 2 provides a summary of the results of an experiment carried out using an incubation step, and no incubation step with an amino acid compound as a nitrogen source. The incubation step was carried out for the first (C-44) yeast samples at 27° C. for 24 hours followed by autolysis at 50° C. for 24-48 hours. The incubation step was skipped for the second (C-47) yeast samples which were put directly into 50° C. for 24-48 hours.

TABLE 2
Testing results from experiments of yeast which has been incubated and
yeast which has not been incubated using amino acid as feed additives

| | | | | Theoretical N % |
|---|---|---|---|---|
| | | | | in 100% sample |
| | | N % 3rd | N % in 100% | (Feeds + yeast), |
| Sample | | Party | Sample | (500 g assumed |
| ID | Feed Additives | Results | (Reported) | as 100% sample) |
| C-47 | Non-incubated yeast (Autolyzed yeast | 1.57 | 0.61 | 1.44 |
| | with 10 g sucrose + 12.5 g Lysine | | | |
| | HCl + 12.5 g Glycine | | | |
| C-44 | Incubated Yeast with 10 g sucrose + | 2.47 | 0.94 | 1.44 |
| | 12.5 g Lysine HCl + 12.5 g Glycine | | | |
| C-42 | Positive control: 10 g sucrose | 1.45 | 0.49 | |

The results indicate that a live yeast according to a preferred embodiment of the present invention would result in the extraction and concentration of nitrogen from an amino acid additive. The nitrogen is in the liquid and therefore can readily be used as a liquid fertilizer or in combination with other compounds to prepare a liquid fertilizer.

Table 3 provides a summary of the results of an experiment carried out using live yeast with amino acid compounds as a nitrogen source with differing carbohydrate inputs. After initial addition of feeds, samples were incubated at 27° C. for 24 hours followed by autolysis at 50° C. for 24-48 hours.

TABLE 3
Testing results from experiments of live yeast using amino
acid as feed additives and comparing the carbohydrate input

| | | | | Theoretical N % |
|---|---|---|---|---|
| | | | | in 100% Sample |
| | | N % 3^{rd} | N % in 100% | (Feeds + yeast), |
| Sample | | Party | Sample | (500 g assumed as |
| ID | Feed Additives | Results | (Reported) | 100% sample) |
| C-42 | Positive control: 5 g | 1.45 | 0.49 | |
| | sucrose initially + 5 g | | | |
| | sucrose next morning | | | |
| C-43 | 10 g Sucrose initially + | 2.11 | 0.89 | 1.44 |
| | 12.5 g Lysine HCl next | | | |
| | morning + 12.5 g | | | |
| | Glycine next morning | | | |
| C-44 | 5 g sucrose initially + | 2.47 | 0.94 | 1.44 |
| | 5 g sucrose next morning + | | | |
| | 12.5 g Lysine HCl | | | |
| | next morning + 12.5 g | | | |
| | Glycine next morning | | | |
| C-75 | Positive control: 5 g | 1.06 | 0.48 | |
| | sucrose initially + 5 g | | | |
| | sucrose next morning | | | |
| C-85 | 10 g sucrose initially + | 1.81 | 0.67 | 0.84 |
| | 6.25 g Lysine sulfate | | | |
| | initially + 6.25 g Lysine | | | |
| | sulfate next morning | | | |
| C-86 | 10 g sucrose initially + | 1.63 | 0.66 | 0.84 |
| | 6.25 g Lysine sulfate | | | |
| | initially + 6.25 g Lysine | | | |
| | sulfate next morning | | | |
| C-87 | 10 g sucrose initially + | 1.74 | 0.63 | 0.84 |
| | 6.25 g Lysine sulfate | | | |
| | initially + 6.25 g Lysine | | | |
| | sulfate next morning | | | |
| C-88 | 5 g sucrose initially + 5 | 1.45 | 0.66 | 0.84 |
| | g sucrose next morning + | | | |
| | 6.25 g Lysine sulfate | | | |
| | initially + 6.25 g lysine | | | |
| | sulfate next morning | | | |
| C-89 | 5 g sucrose + 6.25 g | 1.42 | 0.62 | 0.84 |
| | Lysine sulfate (70%, | | | |
| | 30% carbs) in + 6.25 g | | | |
| | lysine sulfate + 5 g | | | |
| | sucrose nm | | | |
| C-90 | 5 g sucrose + 6.25 g | 1.46 | 0.68 | 0.84 |
| | Lysine sulfate (70%, | | | |
| | 30% carbs) in + 6.25 g | | | |
| | lysine sulfate + 5 g | | | |
| | sucrose nm | | | |

The above results indicate that a live yeast, when batch fed the carbohydrate source, does not promote higher N in the final product. Although the results did not show any significant differences between carbohydrate feeding methods, it was visually evident during the feeding that the yeast had increased activity after the second round of carbohydrate feeding. It is possible that batch feeding the carbohydrate may increase the microbial activity and metabolism and may shorten the incubation time needed to reach the desired result.

Tables 4 relate to an experiment carried out using live yeast with amino acids as a nitrogen source with differing carbohydrate inputs and differing source of amino acid at room temperature. After initial addition of feeds, samples were incubated at 27° C. for 24 hours followed by autolysis at 50° C. for 24-48 hours.

TABLE 4
Results of experiments carried out with samples with live yeast
using amino acids as nitrogen sources and comparing input methods

| | | | | Theoretical N % |
|---|---|---|---|---|
| | | | | in 100% Sample |
| | | N % 3^{rd} | N % in 100% | (Feeds + yeast), |
| | | Party | Sample | (500 g assumed as |
| Sample ID | Feed Additives | Results | (Reported) | 100% sample) |
| C-42 | Positive control: 5 g | 1.45 | 0.49 | |
| | sucrose initially + 5 g | | | |
| | sucrose next morning | | | |
| C-44 | 5 g sucrose initially + | 2.47 | 0.94 | 1.44 |
| | 5 g sucrose next morning + | | | |
| | 12.5 g Lysine HCl | | | |
| | next morning, 12.5 g | | | |
| | Glycine next morning | | | |
| C-45 | 5 g sucrose initially + | 2.5 | 0.82 | 1.44 |
| | 5 g sucrose next morning + | | | |
| | 12.5 g Lysine HCl | | | |
| | initially + 12.5 g | | | |
| | Glycine initially | | | |
| C-46 | 5 g sucrose initially + | 3.12 | 0.92 | 1.44 |
| | 5 g sucrose next morning + | | | |
| | 6.25 g Lysine HCl | | | |
| | initially + 6.25 g | | | |
| | Glycine initially + 6.25 | | | |
| | g Lysine HCl next | | | |
| | morning + 6.25 g | | | |
| | Glycine next morning | | | |
| C-75 | Positive control: 5 g | 1.06 | 0.48 | |
| | sucrose initially + 5 g | | | |
| | sucrose next morning | | | |
| C-76 | 5 g sucrose + 12.5 g | 1.54 | 0.60 | 0.95 |
| | Glycine initially + 5 g | | | |
| | sucrose next morning | | | |
| C-77 | 5 g sucrose + 12.5 g | 1.66 | 0.73 | 0.95 |
| | Glycine initially + 5 g | | | |
| | sucrose next morning | | | |
| C-78 | 5 g sucrose + 12.5 g | 1.84 | 0.74 | 0.95 |
| | Glycine initially + 5 g | | | |
| | sucrose next morning | | | |
| C-79 | 5 g sucrose + 6.25 g | 1.68 | 0.63 | 0.95 |
| | Glycine initially + 6.25 | | | |
| | g Glycine next morning + | | | |
| | 5 g sucrose next morning | | | |
| C-80 | 5 g sucrose + 6.25 g | 1.43 | 0.60 | 0.95 |
| | Glycine initially + 6.25 | | | |
| | g Glycine next morning + | | | |
| | 5 g sucrose next morning | | | |
| C-81 | 5 g sucrose + 6.25 g | 1.86 | 0.56 | 0.95 |
| | Glycine initially + 6.25 | | | |
| | g Glycine next morning + | | | |
| | 5 g sucrose next morning | | | |
| C-82 | 5 g sucrose + 12.5 g | 1.65 | 0.63 | 0.95 |
| | Glycine next morning + | | | |
| | 5 g sucrose next morning | | | |
| C-83 | 5 g sucrose + 12.5 g | 1.55 | 0.60 | 0.95 |
| | Glycine next morning + | | | |
| | 5 g sucrose next morning | | | |
| C-84 | 5 g sucrose + 12.5 g | 1.88 | 0.73 | 0.95 |
| | Glycine next morning + | | | |
| | 5 g sucrose next morning | | | |

The data in the above table demonstrates batch feeding yeast with the nitrogen source does not promote higher N % in the final product. However, observations of the reaction mixture lead to the suggestion that a batch feeding approach will allow one to reduce the time necessary to achieve a high yield (i.e. a substantial part of the reaction will be completed in a shorter period of time). According to a preferred embodiment of the present invention, a batch feeding approach will allow to achieve a similar yield when compared to the non-batch feeding approach, but in a time period which is 20% shorter. According to a preferred embodiment of the present invention, a batch feeding approach will allow to achieve a similar yield when compared to the non-batch feeding approach, but in a time period which is 10% shorter. According to a preferred embodiment of the present invention, a batch feeding approach will allow to achieve a similar yield when compared to the non-batch feeding approach, but in a time period which is more than 20% shorter.

Table 5 relates to experiments carried out using live yeast with different amino acids as a nitrogen source. All samples were incubated at 27-30° C. for 24 hours followed by autolysis at 50° C. for 24-48 hours.

TABLE 5
Results of experiments carried out with samples with live yeast fed with different
amino acids as nitrogen sources and different amounts of carbohydrates

| | | | | Theoretical N % |
|---|---|---|---|---|
| | | | | in 100% sample |
| | | N % 3rd | N % in 100% | (Feeds + yeast), |
| | | Party | Sample | (500 g assumed as |
| Sample ID | Feed Additives | Results | (Reported) | 100% sample) |
| A-11 | 25 g glucose | 2.24 | 0.747 | |
| A-4 | 25 g glucose + 17.5 g Lysine sulfate 70% | 3.02 | 1.068 | 1.188 |
| A-6 | 25 g glucose + 12.5 g Leucine | 2.23 | 0.653 | 1.014 |
| A-7 | 25 g glucose + 12.5 g Aspartic Acid | 1.84 | 0.353 | 1.010 |
| A-8 | 25 g glucose + 12.5 g Arginine | 2.44 | 0.394 | 1.551 |
| A-9 | 25 g glucose + 12.5 g Glycine | 2.77 | 0.895 | 1.213 |
| A-20 | Positive Control: 25 g glucose | 0.75 | 0.241 | |
| A-16 | 25 g glucose + 12.5 g Glutamine | 1.56 | 0.537 | 0.720 |
| A-17 | 25 g glucose + 12.5 g Glutamic Acid | 0.79 | 0.350 | 0.479 |
| C-8 | Positive control: 5 g glucose | 0.95 | 0.607 | |
| C-1 | 5 g glucose + 20 g Lysine Sulfate (70%) | 3.9 | 0.483 | 1.109 |
| C-14 | 5 g glucose + 12.5 g Histidine | 1.56 | 0.671 | 0.865 |
| C-15 | 5 g glucose + 12.5 g Threonine | 0.97 | 0.404 | 0.485 |
| C-34 | Positive control: 10 g sucrose | 0.84 | 0.40 | |
| C-40 | 10 g sucrose + 12.5 g Arginine | 1.61 | 0.60 | 1.201 |
| C-35 | 10 g sucrose + 12.5 g Lysine HCl | 1.51 | 0.63 | 0.876 |
| C-42 | Positive control: 10 g sucrose | 1.45 | 0.49 | |
| C-48 | 10 g sucrose + 12.5 g Valine | 1.76 | 0.66 | 0.79 |
| C-49 | 10 g sucrose + 12.5 g Asparagine | 1.75 | 0.66 | 1.02 |
| C-50 | Positive control: 10 sucrose | 1.22 | 0.54 | |
| C-57 | 10 g sucrose + 12.5 g Lysine HCl | 1.39 | 0.65 | 1.16 |
| C-58 | 10 g sucrose + 12.5 g Tryptophan | 1.44 | 0.64 | 0.99 |
| C-59 | 10 g sucrose + 12.5 g Methionine | 1.35 | 0.65 | 0.88 |
| C-60 | 10 g sucrose + 12.5 g Phenylamine | 1.31 | 0.62 | 0.87 |
| C-61 | 10 g sucrose + 12.5 g Serine | 1.47 | 0.70 | 0.96 |
| C-62 | 10 g sucrose + 12.5 g Cystine | 1.35 | 0.63 | 0.99 |
| C-63 | 10 g sucrose + 12.5 g Tyrosine | 1.3 | 0.44 | 0.82 |
| C-64 | 10 g sucrose + 12.5 g Alanine | 1.57 | 0.72 | 0.83 |

Testing results from experiments C-47 and C-44 show that that non-incubated yeast, when fed the same sources as live yeast, produces a lower N % results than live yeast.

The results from Table 5 shows that amino acids are efficient to use in combination with a live yeast to generate nitrogen in available form for use a fertilizer. The closer the actual result to the theoretical, the better it is as a feed. For example, aspartic acid actual nitrogen concentration was only about 30% of the theoretical so it was not optimally digested by the yeast. On the other hand, glycine was quite close to the theoretical value, which is a clear indication that it was being used by the yeast. There are some fluctuations with the same amino acid depending on the batch of yeast which explains why some of the repeated tests provided different results.

A composition according to a preferred embodiment of the present invention, can be prepared in the absence of a high energy process, since the production of the fertilizer does not require high temperatures. Preferably, the composition utilizes underutilized organic waste product since what is used is a commonly produced waste product that currently has very limited applications or is disposed of, causing problems to municipalities.

According to a preferred embodiment of the present invention, there is a use for the waste produced by this process since it can be recycled into the process or used as a solid fertilizer. According to a preferred embodiment of the present invention, the composition meets Organic Materials Review Institute (OMRI) requirements since ingredients and product can all come from organic origin.

According to a preferred embodiment of the present invention, the liquid fertilizer is organic and meets a 5-1-1 N-P-K content. It is a brown liquid with a pH ranging from 5.0-7.0 and has a specific gravity of 1.2. Preferably, the nutrient content comprises: nitrogen (5.0-6.0 wt. %); phosphorous (0.8-1.5 wt. %); potassium (0.8-1.5 wt. %); and sulfur (0.2-1.0 wt. %).

The above composition (nitrogen (5.0-6.0 wt. %); phosphorous (0.8-1.5 wt. %); potassium (0.8-1.5 wt. %); and sulfur (0.2-1.0 wt. %) obtained according to a preferred embodiment of a process of the present invention was tested against a fish-based fertilizer and used to fertilize several plants. Three different species of plants were grown to maturity: peas, basil, and eggplants. Three groups were observed for each series of testing: a control group (no fertilizer); a group using a composition obtained by a process according to a preferred embodiment of the present invention, and a competitor liquid organic fertilizer (fish-based).

Total Kjeldahl nitrogen on leaves and plant biomass analysis (roots and shoot, wet and dry) was measured in the course of the testing.

### Fertilizer Efficacy Testing on Pea Plants

Several pea plants were fertilized and grown for a period of 50 days under the same conditions, soil, humidity, etc. Upon reaching maturity, visual inspection of the plants indicated that the plants from the control group were chlorotic (leaves turning yellow). Chlorosis is a classic sign of nitrogen deficiency. No visible difference was observed between the two fertilizer treatments.

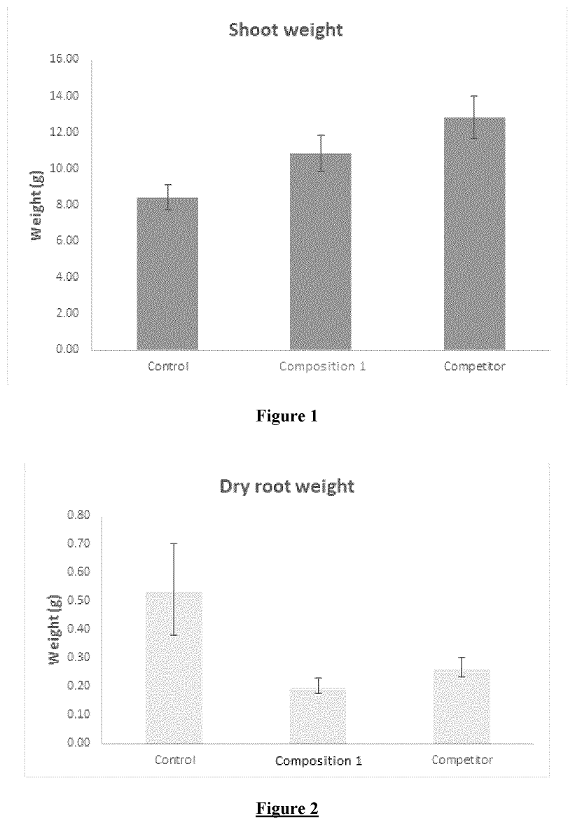

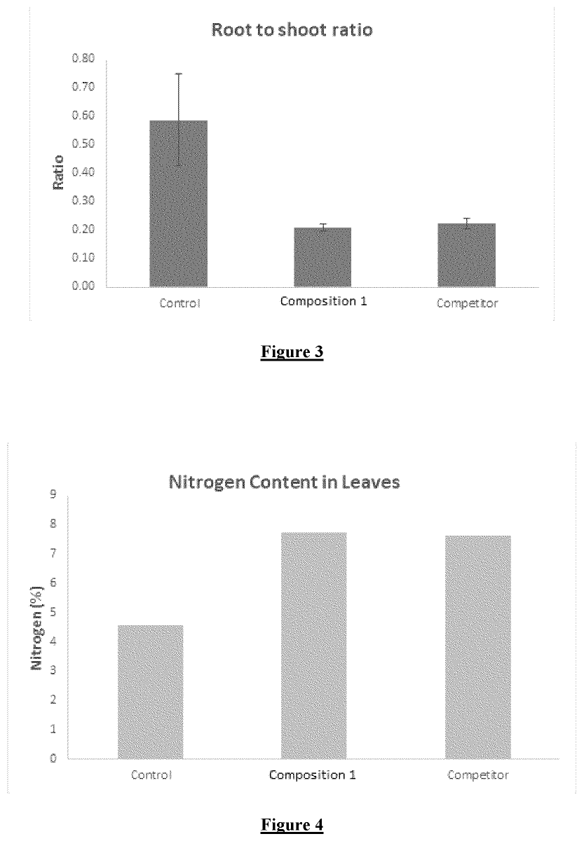

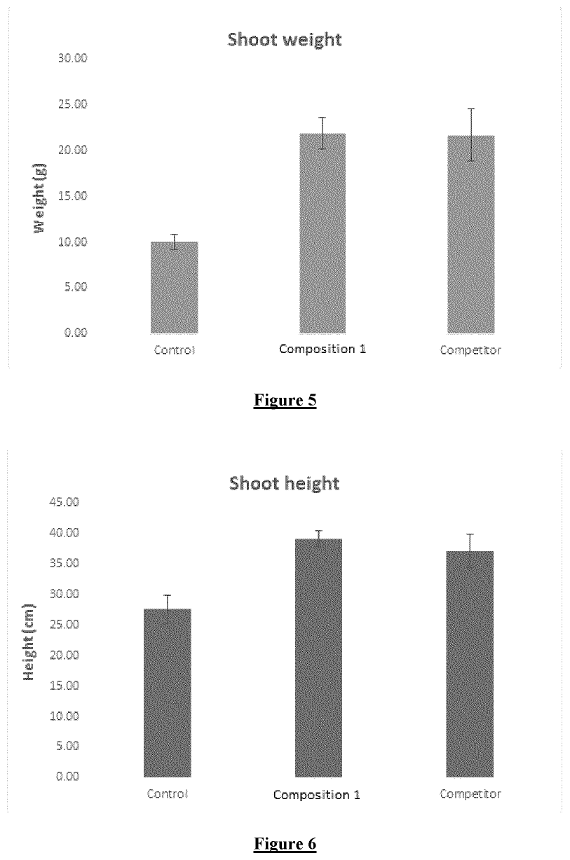

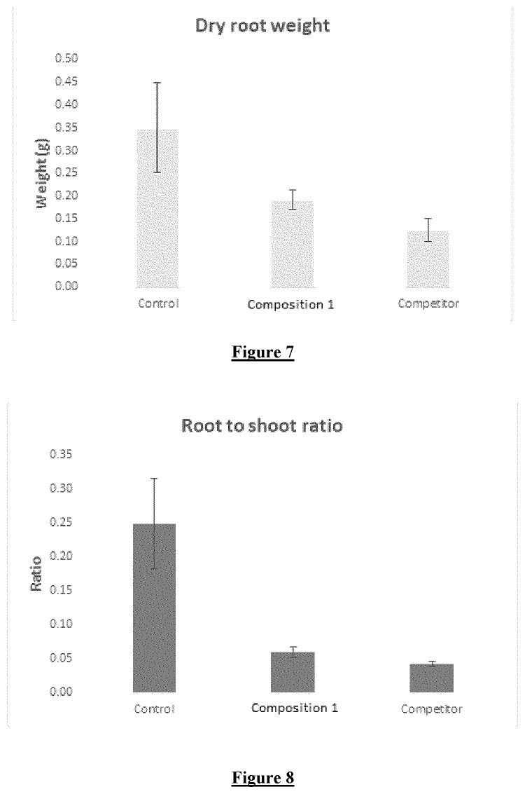

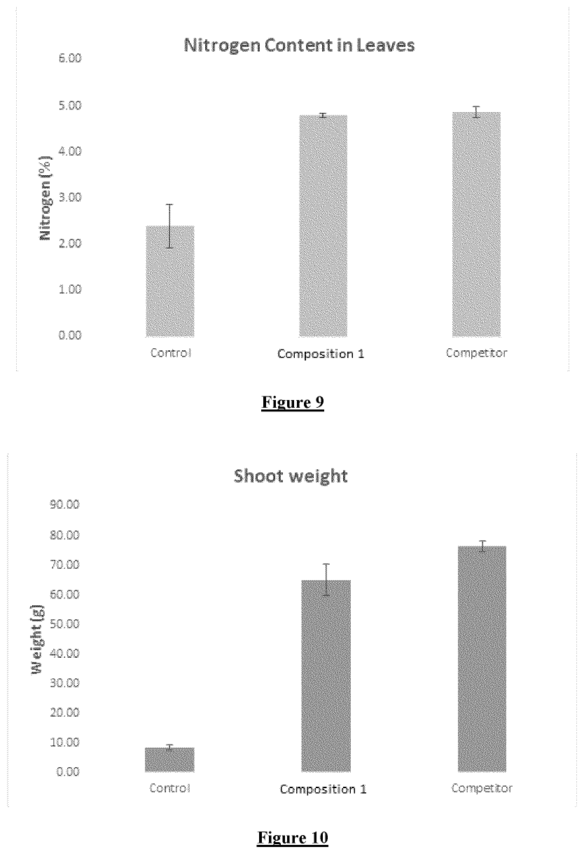

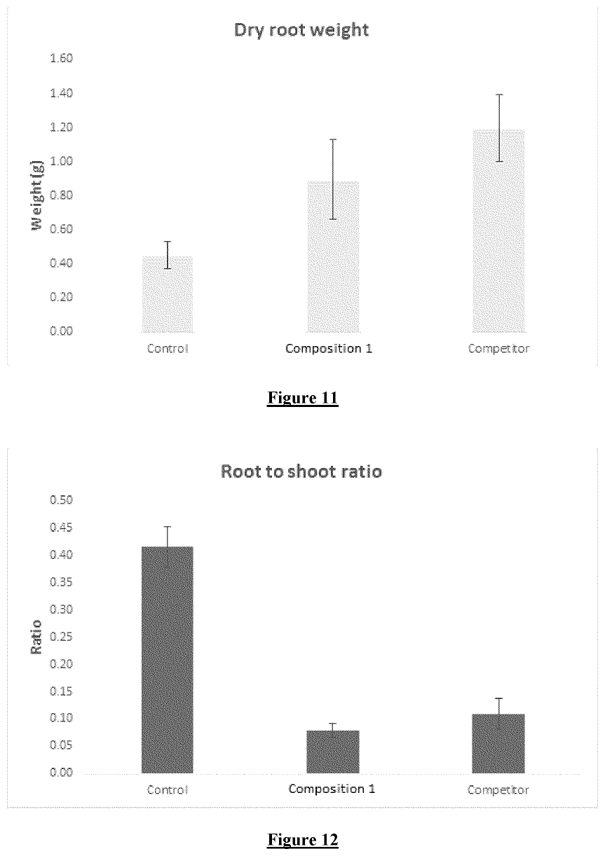

In referring to FIG. 1 one finds a graphical representation of the shoot weight of pea plants (control group, treated fish fertilizer group and treated with a composition obtained according to a preferred embodiment of a process of the present invention, hereinafter referred to as Composition #1). Shoot weight analysis shows smaller shoots in the control plants, suggesting stunted growth due to nutrient deficiency.

In referring to FIG. 2 one finds a graphical representation of the dry root weight of pea plants (control group, treated fish fertilizer group and treated with Composition #1. The roots of the control plants were significantly larger than those of the fertilized plants. This is due to the plant having a nutrient deficiency and putting more energy towards expanding the root system to find nutrients.

In referring to FIG. 3 one finds a graphical representation of the root to shoot ratio of pea plants (control group, treated fish fertilizer group and treated with Composition #1). The root to shoot ratio accounts for any stunted shoot growth and more accurately demonstrates the difference in root growth between treatments. The root to shoot ratio was calculated using wet weights.

In referring to FIG. 4 one finds a graphical representation of the nitrogen content in leaves of pea plants (control group, treated fish fertilizer group and treated with Composition #1). The total Kjeldahl nitrogen in the leaves of the control plants is less than in the treated plants. Pea plants are successfully taking up nitrogen from the fertilizer composition according to a preferred embodiment of the present invention at a similar efficacy as the competitor fertilizer.

### Fertilizer Efficacy on Basil Plants

Several basil plants were fertilized and grown for a period of 72 days under the same conditions, soil, humidity, etc. Upon reaching maturity, visual inspection of the plants indicated that the plants from the control group were smaller, with less leaves, and leaves that showed signs of chlorosis. There were no visible differences between fertilizer treatments.

In referring to FIG. 5 one finds a graphical representation of the shoot weight of basil plants (control group, treated fish fertilizer group and treated with Composition #1). In terms of shoot weight and height, the plants from the control group showed the smallest shoots; which is a sign of stunted growth. This indicates a nutrient deficiency.

In referring to FIG. 6 one finds a graphical representation of the shoot height of basil plants (control group, treated fish fertilizer group and treated with Composition #1). Both groups of fertilized plants showed similar shoot heights and weights, displaying a similar uptake of the fertilizer and similar growth patterns.

In referring to FIG. 7 one finds a graphical representation of the dry root weight of basil plants (control group, treated fish fertilizer group and treated with Composition #1). Root weight and root to shoot ratio is highest in the control plants; which indicates the plants were nutrient deficient and putting their energy into building the root system to find nutrients.

In referring to FIG. 8 one finds a graphical representation of the root to shoot ratio of basil plants (control group, treated fish fertilizer group and treated with Composition #1). Both fertilized group of plants showed similar root to shoot ratio, displaying an efficient uptake of both fertilizers.

In referring to FIG. 9 one finds a graphical representation of the nitrogen content in leaves of basil plants (control group, treated fish fertilizer group and treated with Composition #1). Nitrogen concentration is lowest in the control plants and similar in both fertilizer treatments. The plants were able to take up the nitrogen in Composition #1 at a similar efficacy as the competitor fertilizer.

### Fertilizer Efficacy Testing on Eggplants

Several eggplants were fertilized and grown for a period of 86 days under the same conditions, soil, humidity, etc. Upon reaching maturity, visual inspection of the plants indicated that the plants from the control group showed very stunted growth and severe nutrient deficiency. This is expected as eggplants are known to have a high nitrogen demand. Conversely, both of the fertilizer treatment groups showed similar growth of the plants.

In referring to FIG. 10 one finds a graphical representation of the shoot weight of eggplants (control group, treated fish fertilizer group and treated with Composition #1). Shoot weight is significantly higher in both fertilizer treatments compared to the control. This reflects the severely stunted growth of the control group plants with a nutrient deficiency.

In referring to FIG. 11 one finds a graphical representation of the dry root weight of eggplants (control group, treated fish fertilizer group and treated with Composition #1). Dry root weights were higher in fertilizer treatments compared to the control due to the severely stunted growth in the control group.

In referring to FIG. 12 one finds a graphical representation of the root to shoot ratio of eggplants (control group, treated fish fertilizer group and treated with Composition #1). Higher root to shoot ratio in the controls suggests the plants were nutrient deficient and putting more energy towards the root system to find nutrients. The root to shoot ratio was similar between fertilizer treatment groups.

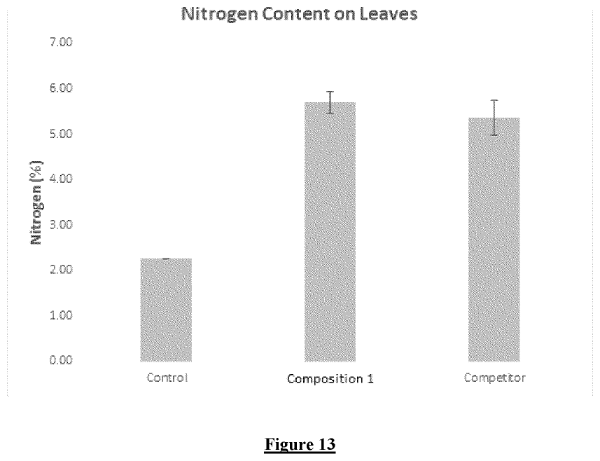

In referring to FIG. 13 one finds a graphical representation of the nitrogen content in leaves of eggplants (control group, treated fish fertilizer group and treated with Composition #1). Nitrogen content is significantly higher in both fertilizer treatments compared to the control group with no significant difference between fertilizer treatment groups.

According to a preferred embodiment of the present invention, the novel process for direct addition and impregnation of nitrogen and sugar sources for the production of both organic liquid and solid fertilizers. This process is robust and able to enrich the nitrogen content of the fertilizer to values ranging from 1 to 8%. This process produces no waste and converts all feed materials into valuable environmentally friendly liquid and solid fertilizers. Preferably, the process starts by obtaining the BSY waste product from a brewery to utilize the yeast and starchy wort for the incubation stage. This stage is followed by the growth and multiplication of yeast in which it multiplies under aerobic conditions to reduce ethanol production and increase cell growth in the presence of a novel drop-in technique of adding sugar and nitrogen sources (such as amino acids). Finally, the growth reaches the targeted stage, the whole broth including yeast is heated to 45° C. for 24 hours.

According to a preferred embodiment of the present invention, the process produces a two-phase broth (Liquid/Solid) which is separated where the liquid is concentrated through evaporation and yielded the liquid fertilizer with a nitrogen content of 1-8%. The solid is sent to a rotary dryer unit and yields a solid organic fertilizer flake.

According to a preferred embodiment of the present invention, the carbohydrate added to the yeast and toe the at least one nitrogen-containing compound can be added in batches, or in continuously or almost continuously fashion using a drip method or the like.

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

1. A process for making a nitrogen-enhanced yeast-based fertilizer, said process comprising the steps of:
a) providing a live yeast Brewer's Spent Yeast (BSY) in a solution;
b) exposing said live yeast to a nitrogen source and to a carbohydrate, thereby creating an incubation mixture;
c) injecting air into the incubation mixture to inhibit the production of ethanol;
wherein said incubation mixture undergoes incubation for a period of time sufficient for said yeast to metabolize said nitrogen source and for said yeast to propagate and store the nitrogen resulting in a nitrogen-fed yeast mixture; and
(d) hydrolyzing (or autolyzing) the resulting nitrogen-fed yeast mixture
wherein the hydrolyzing (or autolyzing) is optionally followed by dehydrating or evaporating.

2. The process according to claim 1, wherein the nitrogen source is a nitrogen-containing compound is selected from any naturally occurring organic protein source.

3. The process according to claim 2, wherein the nitrogen-containing compound is an amino acid selected from Lysine (Lys), Methionine (Met), Tryptophan (Trp), Arginine (Arg), Histidine (His), Isoleucine (Ile), Leucine (Leu), Phenylalanine (Phe), Threonine (Thr), Valine (Val), Glycine (Gly), Cystine (Cys), Tyrosine (Tyr), Alanine (Ala), Glutamine (Gln), Glycine (Gly), Serine (Ser), Asparagine (Asn), Aspartic Acid (Asp), and Glutamic Acid (Glu), combinations thereof, and salts thereof.

4. The process according to claim 1, wherein the incubation mixture undergoes incubation for a period of time ranging from 12 to 48 hours.

5. The process according to claim 1, wherein the incubation mixture undergoes incubation for a period of time ranging from 20 to 36 hours.

6. The process according to claim 1, wherein the nitrogen-enhanced yeast-based fertilizer has a feed rate and a feed quantity that varies vary for each individual fertilizer product.

7. The process according to claim 1, wherein the nitrogen source added to the live yeast in the solution comprises between 0.5 and 20% of the total composition.

8. The process according to claim 1, wherein the nitrogen source added to the live yeast in the solution comprises between 0.5 and 15% of the total composition.

9. The process according to claim 1, wherein the nitrogen source added to the live yeast in the solution comprises between 0.5 and 10% of the total composition.

10. The process according to claim 1, wherein the yeast dry content comprises 0-30 wt. % of the total composition.

11. The process according to claim 1, where wherein the yeast content comprises 7-13 wt. % of the total composition.

12. An enhanced yeast extract, wherein the enhanced yeast extract is prepared by a process comprising a step of exposing yeast to a nutrient prior to a lysis of said yeast.

13. (canceled)

14. A fertilizer comprising a yeast extract enhanced with the addition of at least one nitrogen-containing compound prior to a yeast incubation step, wherein the yeast incubation step is carried out under aerobic conditions.

15. The fertilizer according to claim 14, wherein the nitrogen-containing compound is an inorganic amine salt.

16. The fertilizer according to claim 14, wherein the nitrogen-containing compound is an amino acid selected from alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, combinations thereof, and salts thereof.

17. The fertilizer according to claim 14, comprising between 1 and 7 wt. % of nitrogen.

18. The process according to claim 2, wherein the nitrogen source is a nitrogen-containing compound selected from amino acids, peptides, mixtures of amino acids and/or peptides, and amine salts thereof.
